(19) United States
(12) Patent Application Publication
(10) Pub. No.: US 2024/0181203 A1
(43) Pub. Date: Jun. 6, 2024

(54) Systems and Methods for Clearing Indwelling Catheters

(71) Applicants: University of Washington, Seattle, WA (US); Seattle Children's Hospital d/b/a Seattle Children's Research Institute, Seattle, WA (US)

(72) Inventors: Pierre D. Mourad, Seattle, WA (US); Jason Scott Hauptman, Seattle, WA (US)

(21) Appl. No.: 18/553,610

(22) PCT Filed: Apr. 1, 2022
(86) PCT No.: PCT/US2022/023023

§ 371 Date: Oct. 2, 2023

Related U.S. Application Data
(60) Provisional application No. 63/170,376, filed on Apr. 2, 2021.

(51) Int. Cl. A61M 25/00; A61B 5/06

(52) U.S. Cl. CPC A61M 25/0017; A61B 5/062; A61M 2025/0019

(57) ABSTRACT

Systems, devices, and methods for clearing biodebris or preventing buildup of biodebris in an indwelling catheter. In an example, a device includes a guidance device configured to transcutaneously detect the indwelling catheter implanted in a patient. The device further includes a transducer configured to transcutaneously transmit an ultrasonic pulse to a reservoir at a proximal end of the indwelling catheter. The device additionally includes a controller communicatively coupled to the transducer and configured to adjust one or more settings of the transducer.

### CROSS-REFERENCE TO RELATED APPLICATIONS

The present application is a non-provisional application claiming priority to U.S. Provisional Patent Application No. 63/170,376, filed Apr. 2, 2021, the contents of which are hereby incorporated by reference.

### FIELD OF THE DISCLOSURE

The present disclosure relates to systems and methods of clearing and/or preventing buildup of biodebris in an indwelling catheter. More particularly, a transducer is used to transmit an ultrasonic pulse transcutaneously into the indwelling catheter. The ultrasonic pulse imparts sufficient momentum into the fluid within the catheter that the resulting fluid flow and associated shear stress dislodges and/or prevents from adhering, and clears biodebris from the catheter.

### BACKGROUND

Unless otherwise indicated herein, the materials described in this section are not prior art to the claims in this application and are not admitted to be prior art by inclusion in this section.

An indwelling catheter is a catheter that can be left inside a patient's body, either temporarily or permanently. Indwelling catheters often include one or more pores which can often lead to the buildup of cells and proteins (i.e., “biodebris”). This buildup of biodebris is known as biofouling. In practice, indwelling catheters are likely to clog from biofouling often within the first year of implantation.

Because indwelling catheters are implanted inside a patient's body (e.g., in the brain) for a prolonged period of time, they cannot readily be accessed without an invasive procedure. As such, a clogged indwelling catheter requires an invasive surgical procedure to remove it and replace it. The surgical procedure to remove the indwelling catheter and replace it with a new indwelling catheter is costly and can be damaging to tissue surrounding the indwelling catheter. Moreover, the newly implanted indwelling catheter suffers the same risk of biofouling as the one it replaced.

### SUMMARY

Example devices described herein relates to systems and methods of clearing and/or preventing buildup of biodebris in an indwelling catheter. More particularly, a transducer is utilized to transmit an ultrasonic pulse and/or wave through the skin, or transcutaneously, into the indwelling catheter. The ultrasonic pulse and/or wave then imparts sufficient momentum into the fluid within the catheter that the resulting fluid flow and associated shear stress dislodges and/or prevents from adhering, and clears biodebris from the catheter.

Thus, in one aspect, a device is provided for preventing build-up of biodebris or clearing biodebris from an indwelling catheter which includes a non-invasive guidance device (for example, ultrasound imaging or non-imaging, near-infrared imaging or non-imaging, other light imaging or non-imaging, some but not necessarily all examples) configured to transcutaneously detect the indwelling catheter and/or its reservoir implanted in a patient, henxe guide the focused ultrasound to it, because of the differential reflectance of the combination of skin and underlying reservoir relative to the combination of skin and underlying skull. The device further includes a transducer configured to transcutaneously transmit an ultrasonic wave to a reservoir at a proximal end of the indwelling catheter whose focus is designed to occur in a known position relative to the guidance device. And the device includes a controller communicatively coupled to the transducer and configured to adjust one or more settings of the transducer.

In a second aspect, the device may further or instead include a transducer configured to transcutaneously transmit an ultrasonic wave to a reservoir at a proximal end of the indwelling catheter. The device may additionally include a magnet, magnetometer, electrical impedance measurer, or other non-ultrasound or non-photon-based means of detecting the reservoir, adjacent to the transducer, configured to transcutaneously detect the indwelling catheter and guide the focused ultrasound to the catheter. The device may further include a controller communicatively coupled to the transducer and configured to adjust one or more settings of the transducer.

In another aspect, a method for clearing biodebris from an indwelling catheter or preventing build up biodebris in an indwelling catheter. The method includes detecting, via an imaging or non-imaging guidance device, an indwelling catheter implanted in a patient, wherein the indwelling catheter is detected transcutaneously, hence guides the focused ultrasound to the catheter. The method further includes aligning a transducer with a reservoir on a proximal end of the indwelling catheter. The method additionally includes transmitting, via the transducer, an ultrasonic wave into the reservoir of the indwelling catheter, wherein the ultrasonic wave is transmitted transcutaneously into the reservoir and generates a minimum fluid flow (known as acoustic streaming) within the reservoir and adjacent catheter lumen such that the associated shear stress thereby generated within the indwelling catheter is sufficient to clear the biodebris from within the lumen of the catheter, including its pores, or to prevent the buildup of biodebris at those same locations.

In a further aspect, a system includes an indwelling catheter implanted in a patient including a reservoir at a proximal of the indwelling catheter, and pores at a distal end of the catheter. The system further includes a transducer, configured to transcutaneously detect the indwelling catheter and configured to transcutaneously transmit an ultrasonic wave to the reservoir. The system additionally includes a controller communicatively coupled to the transducer and configured to adjust one or more settings of the transducer.

In yet another aspect, an indwelling catheter device includes pores at a first end of the indwelling catheter device. The indwelling catheter device further includes a reservoir at the second end of the indwelling catheter device. The indwelling catheter device additionally includes a transducer adjacent to the reservoir and configured to transmit an ultrasonic wave into the reservoir, wherein the ultrasonic wave imparts a minimum fluid flow within the catheter such that the resulting shear stress within the indwelling catheter removes biodebris from the pores and propels the biodebris out of the pores or prevents the buildup of biodebris within the catheter.

These as well as other aspects, advantages, and alternatives, will become apparent to those of ordinary skill in the art by reading the following detailed description, with reference where appropriate to the accompanying drawings.

### BRIEF DESCRIPTION OF THE FIGURES

The above, as well as additional, features will be better understood through the following illustrative and non-limiting detailed description of example embodiments, with reference to the appended drawings.

FIG. 1 illustrates a schematic of a transducer device and an indwelling catheter, according to an example embodiment.

FIG. 2 illustrates a side view of a transducer device, according to an example embodiment.

FIG. 3 illustrates a bottom view of a transducer device, according to an example embodiment.

FIG. 4 illustrates a schematic of a transducer device with a detection coil and a reservoir of an indwelling catheter, according to an example embodiment.

FIG. 5 illustrates schematic of a transducer device and a reservoir of an indwelling catheter, according to an example embodiment.

FIG. 6 illustrate a schematic of a reservoir of an indwelling catheter, according to an example embodiment.

FIG. 7 illustrates a method, according to an example embodiment.

All the figures are schematic, not necessarily to scale, and generally only show parts which are necessary to elucidate example embodiments, wherein other parts may be omitted or merely suggested.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

As noted above, indwelling catheters are prone to a certain amount of buildup of biodebris. Systems and methods described herein relate to the prevention of biodebris build-up or the clearing of biodebris built up in indwelling catheters.

More particularly, in embodiments of the present disclosure, the systems apply focused ultrasound transcutaneously within the catheter reservoir proximal to the catheter to propel fluid out of the distal end of the catheter in order to, via its shear stress, dislodge and remove biofouling material and/or prevent biofouling material buildup. This is advantageous to prevent the need to replace the indwelling catheter with a new indwelling catheter which, as noted above, is an invasive neurosurgical procedure that can also be costly and may damage tissue surrounding the catheter.

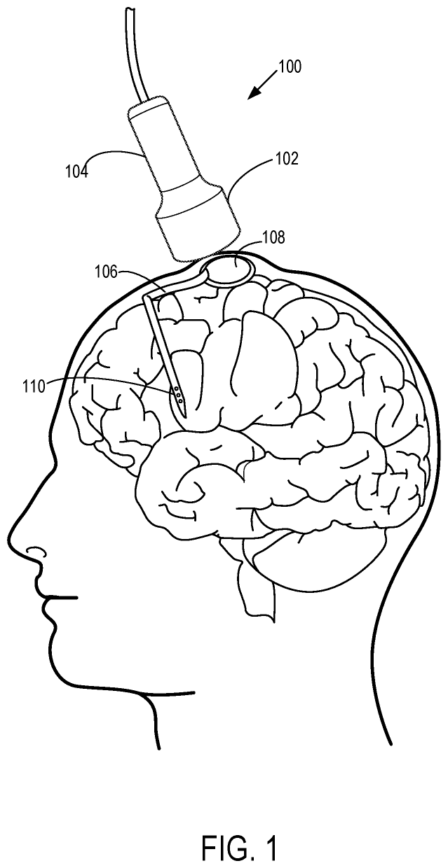

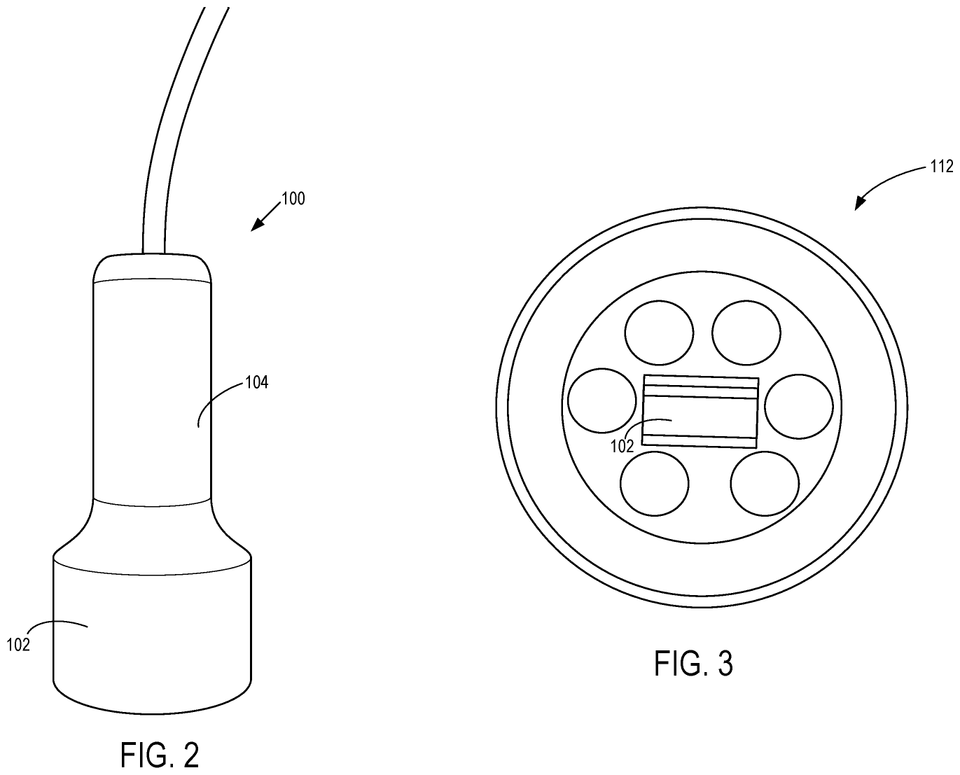

Now referring to FIG. 1, a transducer device 100 and an indwelling catheter 106, according to an example embodiment. In example embodiments, a transducer device 100 includes a transducer 102 at a proximal end of the transducer device 100 and a handle 104. FIGS. 2 and 3 illustrate a side view and a bottom view of an example transducer device 100 according to an example embodiment.

As shown in FIG. 1, an example indwelling catheter 106 includes a reservoir 108 at a proximal end and one or more pores 110 at a distal end. As described in further detail below, transmission of a focused ultrasonic waves can impart application of sufficient shear stress and cause biodebris within the indwelling catheter 106 to propel out of the pores 110. Additionally typically indwelling catheter implanted in a patient's brain includes an additional catheter coupled to the reservoir shown in FIG. 1 which drains spinal fluid often from the brain to the abdomen. These additional catheters are also prone to clogging in a similar manner as described herein. Transmitting an ultrasound from the transducer into the reservoir can also prevent build-up of biodebris and/or help clear a clogged catheter. Note that while FIG. 1 illustrates an indwelling catheter 106 implanted in a patient's brain, systems and methods described herein may be utilized with any indwelling type catheters (e.g., urethra catheter, etc.).

In practice, a user, for example a physician, can first locate the indwelling catheter 106 implanted in the patient utilizing methods outlined in more detail below. Once the indwelling catheter 106 is located, in some examples, the transducer device 100 may have features which can be utilized for aligning the transducer device 100 with the reservoir 108 of the indwelling catheter 106 such that the focal volume lies within the reservoir 108. And once the transducer 102 is aligned with the reservoir 108 of the indwelling catheter 106, an ultrasonic pulse can be transmitted from the transducer 102 to the reservoir 108, generating fluid flow within the reservoir and catheter whose fluid flow and associated shear stress loosens the biodebris then propels that debris along with the fluid out of the pores 110 on the distal end of the indwelling catheter 106, thus preventing the build-up of biodebris and/or clearing the indwelling catheter 106 of biodebris.

As noted above, a user (e.g., a physician) can first locate the indwelling catheter 106 implanted in the patient to align the transducer 102 with the reservoir 108. For example, in some embodiments, the transducer device 100 includes an image guidance device 112. An example guidance device 112 is shown in FIG. 3.

More particularly, in some example embodiments, the guidance device 112 may utilize diagnostic ultrasound. In some examples, the diagnostic ultrasound can include imaging ultrasound. In other examples, the diagnostic ultrasound can include non-imaging ultrasound.

Additionally or alternatively, other guidance technology can be utilized to transcutaneously detect the reservoir 108. For instance, in some examples, the guidance device 112 may include magnetic resonance imaging (MRI). In another example, the guidance device 112 may utilize fluoroscopy. In yet another example embodiment, the guidance device 112 may incorporate a near-infrared spectroscopy system to transcutaneously detect the reservoir 108. Many examples of image guidance technology are possible.

Regardless of the guidance technology, in example embodiments, the guidance device 112 may provide detection data to a device (e.g., an ultrasound machine, a computer, a smartphone, etc.) in order to provide real-time positional feedback to the physician. Thus, the physician can align the transducer 102 with the reservoir 108.

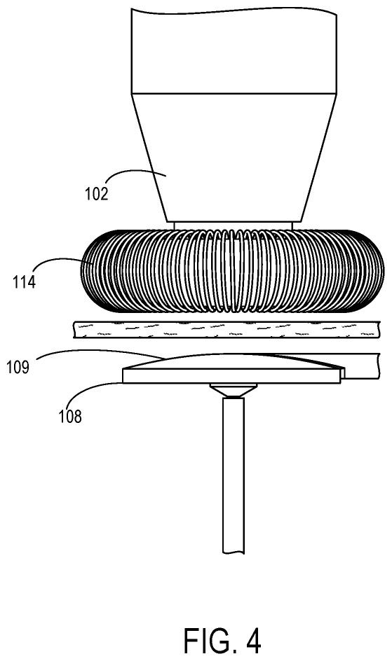

Now referring to FIG. 4, a schematic of a transducer device 100 with a detection coil 114 and a reservoir 108 of an indwelling catheter, according to an example embodiment. In some examples, the transducer device 102 may include a detection coil 114 to locate and align the transducer 102 with the reservoir 108, such that the focal volume of the transducer 102 is within the volume of the reservoir 108 and the focal point is directed to the center of the reservoir 108. Namely, the detection coil 114 may surround the transducer 102 at the proximal end of the transducer device 100.

In these examples, the reservoir 108 may include a metal or plastic component or element. And the transducer device 102 can include an electromechanical, ultrasound, or light-based guidance process to locate and/or detect the reservoir 108 and thus enable alignment of the focus of the transducer 102 with the reservoir 108.

In examples, where the reservoir 108 includes a metal component, a magnetic detection coil 114 may be utilized to detect the reservoir 108. In examples where there reservoir 108 includes a plastic component, the detection coil 114 may utilize electrical impedance detection to detect the reservoir 108.

In some example embodiments, the transducer device 102 does not incorporate external-energy (ultrasound, light, electromagnetism) guidance, instead it utilizes anatomical guidance. For example, detection of the bump under the skin generated by the presence of the reservoir, facilitated by magnetic coupling via the detection coil 114.

Further, some example embodiments include a combination of energy-based guidance technology, described herein, with anatomical guidance and/or magnetic coupling. For instance, an example transducer device can include both a guidance device 112 and/or a detection coil 114.

Once the transducer device 100 has detected the indwelling catheter 106 and is aligned such that the focal volume of the transducer 102 is directed within the volume of the reservoir 108, in some embodiments, the transducer device 100 includes a feature to stabilize the transducer 102 in the aligned position. For instance, in some examples, the transducer device 100 can include a mechanical coupling compatible with a cap 109 of the reservoir 108.

More particularly, the skin of the patient may be raised where the reservoir 108 sits beneath it. As such, the proximal end of the transducer device 100 may be shaped to tightly surround the reservoir 108, providing stability of the alignment of the transducer 102 with the reservoir 108. Other examples of mechanical couplings are possible.

Additionally or alternatively, in some embodiments, the reservoir 108 may be shaped in such a way to be compatible with the transducer device 100. For example, the reservoir 108 may have a similar shape as the proximal end of the transducer device 100.

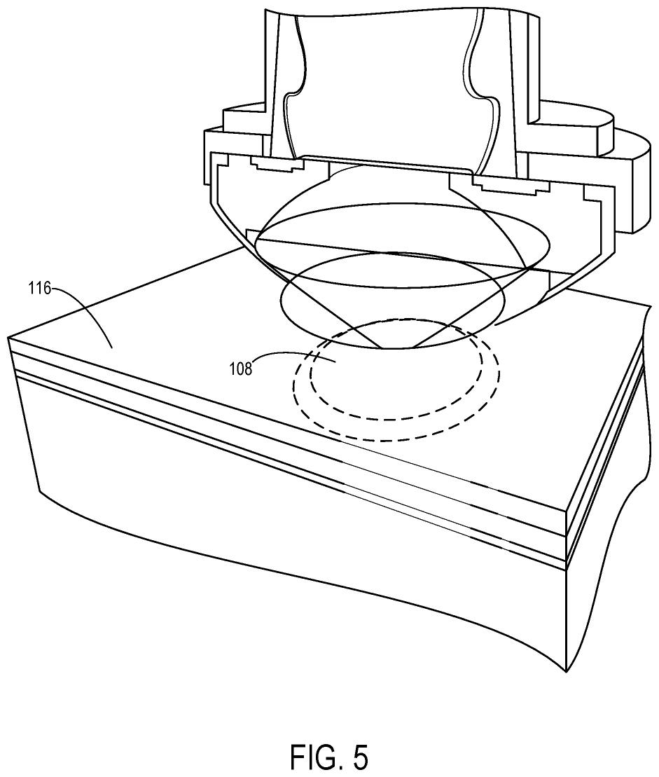

Now referring to FIG. 5, which illustrates the transducer 102 aligned with the reservoir 108, which lies beneath a layer of skin 116 when implanted. Once the transducer 102 is aligned with the reservoir 108, a user (e.g., a physician) can initiate the transducer 102 to transmit an ultrasonic pulse transcutaneously into the reservoir 108 typically by way of a controller communicatively coupled to the transducer 102. Note that the transducer 102 is configured to transmit a series of ultrasonic pulses and/or an ultrasonic wave comprising a series of ultrasonic pulses.

In some example embodiments, the transducer 102 is communicatively coupled to a controller. For instance, the controller may be communicatively coupled to the transducer 102 by way of a wired or wireless connection.

The controller is configured to adjust one or more settings of the transducer and corresponding ultrasonic pulses. For instance, in an example embodiment, the controller can adjust the frequency, power, or focal distance settings of the transducer. This may be advantageous to adjust for variances in the type and condition of the catheter.

More particularly, different types of indwelling catheters (brain, urethra, etc.) will have different specific shapes. The specific shapes of the various catheters require distinct refinements as to type of ultrasound to guide and deliver.

The controller may include a function to initiate the transmission of an ultrasonic pulse into the reservoir 108 of the indwelling catheter 106. More particularly, the transducer transmits ultrasound delivered via the transducer 102 transcutaneously the reservoir 108 of the indwelling catheter 106 such that cerebral spinal fluid biodebris will be pushed back down the indwelling catheter 106 with generation of sufficient shear stress to unblock the catheter.

In some examples, the controller includes at least one processor and data storage including program instructions stored thereon that when executed by the at least one processor, cause the transducer device 102 to perform one or more of the functions described herein.

Different ultrasound protocols can be implemented in various scenarios and/or for different types and sizes of catheters. For example, the frequency, power, focal distance, and other components of the ultrasound can be adjusted for different size catheters. Additionally, the frequency, power, focal distance, and other components of the ultrasound can be adjusted for unclogging a blocked catheter versus prevention of buildup of biodebris, for example.

In some examples, ultrasound protocols can provide a series of chronic ultrasound pulses which are temporally long and provide a constant shear stress. Such an ultrasound protocol can be advantageous as a preventative measure to keep indwelling catheters clear starting soon after catheter implantation. Such anticipated ultrasound protocols can transmit relatively long pulses of relatively low-intensity ultrasound to generate sufficient flow persistently in order to keep cells and proteins from sticking to the catheters. Examples of ultrasound protocols include, but are not limited to: spatial peak, temporal average intensity (I_spta) values ranging between 0.1-10 W/cm2 with carrier frequencies ranging between 0.5-10 MHz. In such protocols, each ultrasound pulse of the series of ultrasound pulses can last for 0.1-0.5 seconds or longer. The ultrasound pulses in these example protocols can be spaced in time by 0.1-0.5 seconds or longer, for example.

Alternatively, an ultrasound protocol can include transmitting many short ultrasonic pulses producing rapidly changing shear stress. These ‘acute’ ultrasound protocols can be utilized to clear a clogged catheters, for example, a typical emergent concern. These anticipated ultrasound protocols will use relatively short pulses of relatively high-intensity ultrasound in order to generate maximum flow within the catheter sufficient to remove the adherent, biofouling material, acutely.

Examples of ultrasound protocols include, but are not limited to: I_spta values ranging between 1.0-1000 W/cm2 with carrier frequencies ranging between 1.0-5.0 MHz. Such protocols can consist of ultrasonic pulses measuring 0.001-0.1 seconds in length. The time between the ultrasonic pulses in such protocols can be 0.001-0.1 seconds, for examples.

In some examples, an ultrasound protocol can involve the combination of long ultrasound pulses and short ultrasound pulses. More particularly, the short ultrasonic pulses (e.g., 0.001-0.1 seconds) may be interleaved between the longer ultrasonic pulses (e.g., 0.1-0.5 seconds). Many examples ultrasound protocols are possible.

Further, ultrasound protocols may use a carrier frequency between 1-10 MHz or higher, with higher frequencies allowing for sufficiently small focal volumes. Namely, the higher frequencies allow the ultrasound pulse to fit within the reservoir 108 while avoiding the overlying skin. In these examples, spatial peak, time-averaged intensity values can range from 10 W/cm2-1000 W/cm2 or higher. Higher spatial peak, time-averaged intensity values may be employed for shorter pulses.

In example embodiments, it is possible to shape of the focus of the focused ultrasound to generate either of chronic or acute focused ultrasound field for indwelling catheters. For example, the more that a spherical, elliptical or parabolic ultrasound source (itself made up of one to many transducers across a range of materials) subtends a sufficient percentage of the ultrasounds' canonical shape, such that ultrasound source will generate a more compact ultrasound focus. Even flat ultrasound arrays can generate a focus of an optimal shape for existing catheters, through use of electronic beamforming.

Additionally or alternatively, the proximal aspect of indwelling catheters 106 and or the reservoir 108 may be designed to optimally receive focused ultrasound in a way that, in turn, generates optimal flow within that distal aspect of the reservoir (at the top of the catheter itself) hence within the catheter itself.

For example, the shape of the reservoir 108 can ensure that a sufficient fluid volume exists within the proximal aspect of the indwelling catheter 106 such that it can receive the focused ultrasound well away from any boundaries that the ultrasound may bounce off of or harm surrounding tissue.

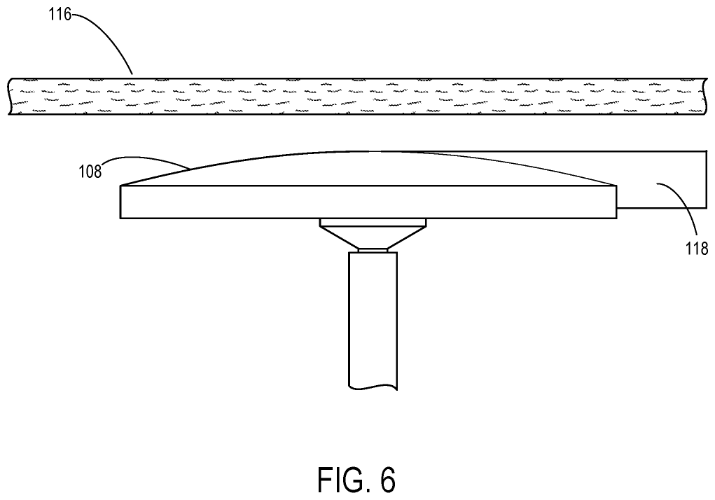

Now referring to FIG. 6, a reservoir 108 of an indwelling catheter device with an ultrasonic transducer system 118 incorporated directly into the catheter reservoir 108. Namely, in some example embodiments an ultrasonic transducer may be implanted in the patient along with the indwelling catheter 106. The ultrasonic transducer may be adjacent to the reservoir so as to transmit an ultrasonic pulse directly into the reservoir 108.

In these example embodiments, systems that power and control the ultrasonic transducer system 118 may do so via transcutaneous wires or wirelessly, with that wireless control and energy facilitated via ultrasound or electromagnetic energy.

Additionally, in these example embodiments, the reservoir 108 itself may incorporate the source of focused ultrasound energy (at least one transducer) such that the focused ultrasound source, powered and controlled transcutaneously and/or wirelessly, propels fluid out of the distal end of the catheter in order to remove the fouling material and/or prevent biofouling material buildup.

Further, in these example embodiments, the ultrasonic transducer system 118 may employ any of the various ultrasound protocols described herein or any combination of the various ultrasound protocols described herein.

Several catheter device and/or ultrasound system embodiments may be combined for use in embodiments of the system described herein. Some indwelling catheter and/or transducer devices may involve different means as well as type of ultrasound delivery, others involving catheter design to receive ultrasound, and appropriate combinations.

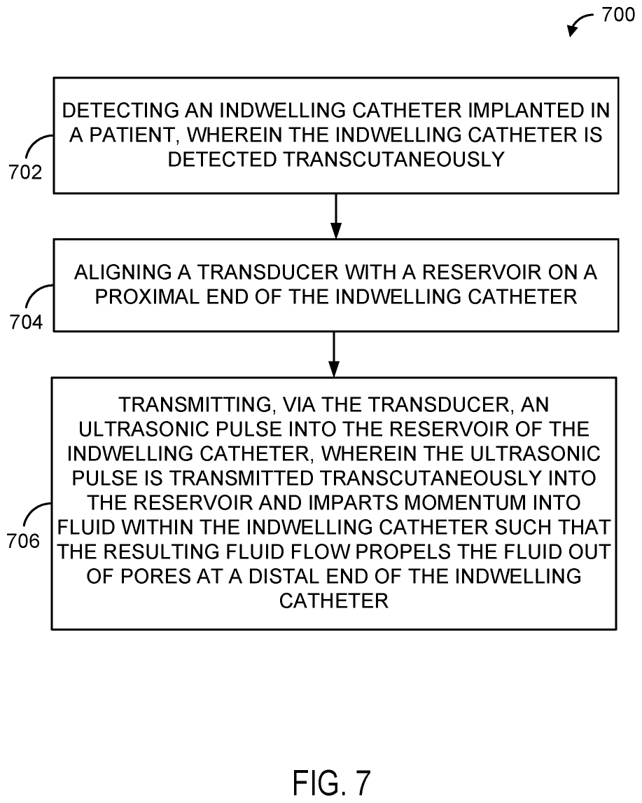

FIG. 7 is a block diagram of an example method for clearing or preventing buildup of an indwelling catheter. Method 700 shown in FIG. 7 presents an embodiment of a method that could be used by the transducer devices and/or indwelling catheter devices described in FIGS. 1-6, as examples. Method 700 may include one or more operations, functions, or actions as illustrated by one or more of blocks 702-706. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

In addition, for the method 700 and other processes and methods disclosed herein, the block diagram shows functionality and operation of one possible implementation of present embodiments. In this regard, each block may represent a module, a segment, or a portion of program code, which includes one or more program instructions executable by a processor or computing device for implementing specific logical functions or steps in the process. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

Initially, at block 702, the method 700 includes detecting, via a guidance device, an indwelling catheter implanted in a patient, wherein the indwelling catheter is detected transcutaneously.

At block 704, the method 700 includes aligning a transducer with a reservoir on a proximal end of the indwelling catheter.

At block 706, the method 700 includes transmitting, via the transducer, an ultrasonic pulse into the reservoir of the indwelling catheter, wherein the ultrasonic pulse is transmitted transcutaneously into the reservoir and imparts a minimum shear stress such that fluid within the indwelling catheter is propelled out of pores at a distal end of the indwelling catheter.

In some example embodiments, the method further includes transmitting, via the transducer, a series of ultrasonic pulses measuring 0.05-1.00 seconds, each of the ultrasonic pulses of the series of ultrasonic pulses spaced apart in time by 0.05-1.00 seconds.

In another example embodiment, the method further includes transmitting, via the transducer, a series of ultrasonic pulses measuring 0.001-0.1 seconds, each of the ultrasonic pulses of the series of ultrasonic pulses spaced apart in time by 0.001-0.1 seconds.

In a further example embodiment, the method further includes transmitting, via the transducer, a first series of ultrasonic pulses measuring 0.05-1.00 seconds, each of the ultrasonic pulses of the first series of ultrasonic pulses spaced apart in time by 0.05-1.00 seconds. In this example, the method may further include transmitting, via the transducer, a second series of ultrasonic pulses interleaved with the first series of ultrasonic pulses, each of the ultrasonic pulses of the second series of ultrasonic pulses measuring 0.05-1.00 seconds and spaced apart in time by 0.05-1.00 seconds.

Each of the transducer devices described in FIGS. 1-5 and/or the indwelling catheter device describe in FIG. 6 may represent a module, a segment, or a portion of program code, which includes one or more instructions executable by a processor or computing device for creating such devices using an additive-manufacturing system. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

### Examples

In an example, device comprising a guidance device configured to transcutaneously detect the indwelling catheter implanted in a patient; a transducer configured to transcutaneously transmit an ultrasonic pulse to a reservoir at a proximal end of the indwelling catheter; and a controller communicatively coupled to the transducer and configured to adjust one or more settings of the transducer.

In an example, the ultrasonic pulse is part of an ultrasonic protocol comprising a series of ultrasonic pulses.

In an example, the ultrasonic protocol comprises a series of ultrasonic pulses measuring 0.05-1.00 seconds.

In an example, each ultrasonic pulse in the series of ultrasonic pulses are spaced apart in time by 0.05-1.00 seconds.

In an example, the ultrasonic protocol comprises a series of ultrasonic pulses measuring 0.001-0.1 seconds in length.

In an example, each ultrasonic pulse in the series of ultrasonic pulses are spaced apart in time by 0.001-0.1 seconds in length.

In an example, the guidance device comprises at least one of magnetic resonance imaging (MRI), diagnostic ultrasound, fluoroscopy, or near-infrared spectroscopy.

An example device comprises a magnet at a distal end of the device.

In an example, the device further comprises a mechanical coupling compatible with a cap of the reservoir.

In an example, the one or more settings of the transducer comprises at least one of a frequency, a power, or a focal distance.

In an example, the controller comprises: at least one processor; and data storage including program instructions stored thereon that when executed by the at least one processor, cause the device to: transmit a series of ultrasonic pulses measuring 0.05-1.00 seconds, each of the ultrasonic pulses of the series of ultrasonic pulses spaced apart in time by 0.05-1.00 seconds.

In an example, the controller comprises: at least one processor; and data storage including program instructions stored thereon that when executed by the at least one processor, cause the transducer to: transmit a series of ultrasonic pulses measuring 0.001-0.1 seconds, each of the ultrasonic pulses of the series of ultrasonic pulses spaced apart in time by 0.001-0.1 seconds.

In an example, a transducer configured to transcutaneously transmit an ultrasonic pulse to a reservoir at a distal end of the indwelling catheter; a magnet, adjacent to the transducer, configured to transcutaneously detect the indwelling catheter; and a controller communicatively coupled to the transducer and configured to adjust one or more settings of the transducer.

In an example, the ultrasonic pulse is part of an ultrasonic protocol comprising a series of ultrasonic pulses.

In an example, the ultrasonic protocol comprises a series of ultrasonic pulses measuring 0.05-1.00 seconds.

In an example, each ultrasonic pulse in the series of ultrasonic pulses are spaced apart in time by 0.05-1.00 seconds.

In an example, the ultrasonic protocol comprises a series of ultrasonic pulses measuring 0.001-0.1 seconds in length.

In an example, each ultrasonic pulse in the series of ultrasonic pulses are spaced apart in time by 0.001-0.1 seconds in length.

In an example, the device further comprises a mechanical coupling compatible with a cap the reservoir.

In an example, the one or more settings of the transducer comprises at least one of the frequency, power, of focal distance.

In an example, a method comprises: detecting, via a guidance device, an indwelling catheter implanted in a patient, wherein the indwelling catheter is detected transcutaneously; aligning a transducer with a reservoir on a proximal end of the indwelling catheter; and transmitting, via the transducer, an ultrasonic pulse into the reservoir of the indwelling catheter, wherein the ultrasonic pulse is transmitted transcutaneously into the reservoir and imparts momentum into fluid within the indwelling catheter such that the resulting fluid flow propels the fluid out of pores at a distal end of the indwelling catheter.

In an example, transmitting, via the transducer, an ultrasonic pulse into the reservoir comprises transmitting a series of ultrasonic pulses, the method further comprising: transmitting, via the transducer, a series of ultrasonic pulses measuring 0.05-1.00 seconds in duration, each of the ultrasonic pulses of the series of ultrasonic pulses spaced apart in time by 0.05-1.00 seconds.

In an example, transmitting, via the transducer, an ultrasonic pulse into the reservoir comprises transmitting a series of ultrasonic pulses, the method further comprising: transmitting, via the transducer, a series of ultrasonic pulses measuring 0.001-0.1 seconds in duration, each of the ultrasonic pulses of the series of ultrasonic pulses spaced apart in time by 0.001-0.1 seconds.

In an example, transmitting, via the transducer, an ultrasonic pulse into the reservoir comprises transmitting a series of ultrasonic pulses, the method further comprising: transmitting, via the transducer, a first series of ultrasonic pulses measuring 0.05-1.00 seconds in duration, each of the ultrasonic pulses of the first series of ultrasonic pulses spaced apart in time by 0.05-1.00 seconds; and transmitting, via the transducer, a second series of ultrasonic pulses interleaved with the first series of ultrasonic pulses, each of the ultrasonic pulses of the second series of ultrasonic pulses measuring 0.05-1.00 seconds in duration and spaced apart in time by 0.05-1.00 seconds.

In an example, a system comprising: an indwelling catheter implanted in a patient comprising a reservoir at a proximal of the indwelling catheter, and pores at a distal end of the catheter; and a transducer, configured to transcutaneously detect the indwelling catheter and configured to transcutaneously transmit an ultrasonic pulse to the reservoir; and a controller communicatively coupled to the transducer and configured to adjust one or more settings of the transducer.

In an example, an indwelling catheter device comprising: pores at a first end of the indwelling catheter device; a reservoir at a second end of the indwelling catheter device; and a transducer adjacent to the reservoir and configured to transmit an ultrasonic pulse into the reservoir, wherein the ultrasonic pulse imparts momentum into fluid within the indwelling catheter such that the resulting fluid flow propels the fluid out of pores at a distal end of the indwelling catheter.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

1. A device comprising:
a guidance device configured to transcutaneously detect an indwelling catheter implanted in a patient;
a transducer configured to transcutaneously transmit an ultrasonic pulse to a reservoir at a proximal end of the indwelling catheter; and
a controller communicatively coupled to the transducer and configured to adjust one or more settings of the transducer.

2. The device of claim 1, wherein the ultrasonic pulse is part of an ultrasonic protocol comprising a series of ultrasonic pulses.

3. The device of claim 2, wherein the ultrasonic protocol comprises a series of ultrasonic pulses measuring 0.05-1.00 seconds in duration.

4. The device of claim 3, wherein each ultrasonic pulse in the series of ultrasonic pulses are spaced apart in time by 0.05-1.00 seconds.

5. The device of claim 2, wherein the ultrasonic protocol comprises a series of ultrasonic pulses measuring 0.001-0.1 seconds in duration.

6. The device of claim 5, wherein each ultrasonic pulse in the series of ultrasonic pulses are spaced apart in time by 0.001-0.1 seconds in duration.

7. The device of claim 1, wherein the guidance device comprises at least one of magnetic resonance imaging (MRI), diagnostic ultrasound, fluoroscopy, or near-infrared spectroscopy.

8. The device of claim 1, further comprising a magnet at a distal end of the device.

9. The device of claim 1, wherein the device further comprises a mechanical coupling compatible with a cap of the reservoir.

10. The device of claim 1, wherein the one or more settings of the transducer comprises at least one of a frequency, a power, or a focal distance.

11. The device of claim 1, wherein the controller comprises:
at least one processor; and
data storage including program instructions stored thereon that when executed by the at least one processor, cause the device to:
transmit a series of ultrasonic pulses measuring 0.05-1.00 seconds in duration, each of the ultrasonic pulses of the series of ultrasonic pulses spaced apart in time by 0.05-1.00 seconds.

12. (canceled)

13. A device comprising:
a transducer configured to transcutaneously transmit an ultrasonic pulse to a reservoir at a distal end of an indwelling catheter;
a magnet, adjacent to the transducer, configured to transcutaneously detect the indwelling catheter; and
a controller communicatively coupled to the transducer and configured to adjust one or more settings of the transducer.

14. The device of claim 13, wherein the ultrasonic pulse is part of an ultrasonic protocol comprising a series of ultrasonic pulses.

15. The device of claim 14, wherein the ultrasonic protocol comprises a series of ultrasonic pulses measuring 0.05-1.00 seconds in duration.

16. The device of claim 15, wherein each ultrasonic pulse in the series of ultrasonic pulses are spaced apart in time by 0.05-1.00 seconds.

17. The device of claim 14, wherein the ultrasonic protocol comprises a series of ultrasonic pulses measuring 0.001-0.1 seconds in duration.

18. The device of claim 17, wherein each ultrasonic pulse in the series of ultrasonic pulses are spaced apart in time by 0.001-0.1 seconds in duration.

19. The device of claim 13, wherein the device further comprises a mechanical coupling compatible with a cap the reservoir.

20. The device of claim 13, wherein the one or more settings of the transducer comprises at least one of the frequency, power, of focal distance.

21. A method comprising:
detecting, via a guidance device, an indwelling catheter implanted in a patient, wherein the indwelling catheter is detected transcutaneously;
aligning a transducer with a reservoir on a proximal end of the indwelling catheter; and
transmitting, via the transducer, an ultrasonic pulse into the reservoir of the indwelling catheter, wherein the ultrasonic pulse is transmitted transcutaneously into the reservoir and imparts momentum into a fluid within the indwelling catheter such that a resulting fluid flow propels the fluid out of pores at a distal end of the indwelling catheter.

22-26. (canceled)
